(19) United States
(12) Patent Application Publication
(10) Pub. No.: US 2025/0169970 A1
(43) Pub. Date: May 29, 2025

(54) STENT WITH ATRAUMATIC ANCHORING

(71) Applicant: BOSTON SCIENTIFIC SCIMED, INC., Maple Grove, MN (US)

(72) Inventor: Martyn G. Folan, Galway (IE)

(73) Assignee: BOSTON SCIENTIFIC SCIMED, INC., Maple Grove, MN (US)

(21) Appl. No.: 18/962,725

(22) Filed: Nov. 27, 2024

Related U.S. Application Data
(60) Provisional application No. 63/603,848, filed on Nov. 29, 2023.

(51) Int. Cl. A61F 2/90; A61F 2/95

(52) U.S. Cl. CPC A61F 2/90; A61F 2/95

(57) ABSTRACT

A medical stent includes an elongate tubular body extending from a proximal end region to a distal end region. The proximal end region defines a radially extending flange that is adapted to secure the elongate tubular body relative to a first bodily structure. The distal end region defines two or more elongate anchors movable from a delivery configuration to a deployment configuration in which the two or more elongate anchors extend radially outwardly from the elongate tubular body, the two or more elongate anchors adapted to secure the elongate tubular body relative to a second bodily structure when in the deployment configuration.

### CROSS REFERENCE TO RELATED APPLICATIONS

The present application claims the benefit of and priority to U.S. Provisional Patent Application Ser. No. 63/603,848, filed on Nov. 29, 2023, the disclosure of which is incorporated herein by reference.

### TECHNICAL FIELD

The present disclosure relates generally to methods and apparatuses for various ailments. More particularly, the disclosure relates to different configurations and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain patency of the structure. These devices are manufactured by any one of a variety of different manufacturing methods and may be used according to any one of a variety of methods for a variety of applications. Of the known medical stents, delivery systems, and methods, each has certain advantages and disadvantages. For example, in some stents, the compressible and flexible properties that assist in stent delivery may also result in a stent that has a tendency to migrate from its originally deployed position in a body lumen. As an example, stents that are designed to be positioned in the esophageal or gastrointestinal tract may have a tendency to migrate due to peristalsis (i.e., the involuntary constriction and relaxation of the muscles of the esophagus, intestine, and colon which push the contents of the canal therethrough). There is an ongoing need to provide alternative medical stents and delivery devices as well as alternative methods for manufacturing and using medical stents and delivery devices, such as those susceptible to migration in the anatomy.

### SUMMARY

The disclosure is directed to several alternative designs, materials and methods of manufacturing medical device structures and assemblies, and the use thereof.

A first example is a medical stent including an elongate tubular body having an intermediate body region extending between a proximal end region and a distal end region. The proximal end region defines a radially extending flange that is adapted to secure the elongate tubular body relative to a first bodily structure. The distal end region defines two or more elongate anchors movable from a delivery configuration to a deployment configuration in which the two or more elongate anchors extend radially outwardly from the elongate tubular body. The two or more elongate anchors are adapted to secure the elongate tubular body relative to a second bodily structure when in the deployment configuration.

Alternatively or additionally to any of the examples above, in another example, the distal end region comprises two or more elongate voids disposed between the two or more elongate anchors.

Alternatively or additionally to any of the examples above, in another example, the deployment configuration represents a remembered configuration for the two or more elongate anchors.

Alternatively or additionally to any of the examples above, in another example, each of the two or more elongate anchors are constrained during delivery to maintain the two or more elongate anchors in the delivery configuration, and each of the two or more elongate anchors are adapted to move into the deployment configuration upon removal of the constraint.

Alternatively or additionally to any of the examples above, in another example, the two or more elongate anchors have a curved profile matching a curved profile of the intermediate body region of the elongate tubular body when in the delivery configuration.

Alternatively or additionally to any of the examples above, in another example, the two or more elongate anchors have a curved profile curving in an opposite direction from a curved profile of the intermediate body region of the elongate tubular body when in the delivery configuration.

Alternatively or additionally to any of the examples above, in another example, the two or more elongate anchors have a planar profile different from a curved profile of the intermediate body region of the elongate tubular body when in the delivery configuration.

Alternatively or additionally to any of the examples above, in another example, at least one of the two or more elongate anchors have a braid pattern different from that of the intermediate body region of the elongate tubular body.

Alternatively or additionally to any of the examples above, in another example, the medical stent further includes a polymeric covering extending over the intermediate body region of the elongate tubular body.

Alternatively or additionally to any of the examples above, in another example, the polymeric covering extends over the at least two elongate anchors.

Alternatively or additionally to any of the examples above, in another example, the polymeric covering extends between the at least two elongate anchors.

Alternatively or additionally to any of the examples above, in another example, the medical stent further includes a second radially extending flange distal of the radially extending flange within the proximal end region.

Alternatively or additionally to any of the examples above, in another example, the elongate tubular body comprises a braided stent body.

Another example is medical stent including an elongate tubular body having an intermediate body region extending between a proximal end region and a distal end region. The proximal end region defines a proximal end anchor structure. The distal end region defines two or more anchors movable from a delivery configuration to a deployment configuration in which the two or more elongate anchors extend radially outwardly from the elongate tubular body.

Alternatively or additionally to any of the examples above, in another example, the proximal end anchor structure comprises a radially extending flange.

Alternatively or additionally to any of the examples above, in another example, the proximal end anchor structure comprises a radially extending flange.

Alternatively or additionally to any of the examples above, in another example, the proximal end anchor structure comprises an additional two or more elongate anchors movable from a delivery configuration to a deployment configuration in which the additional two or more elongate anchors extend radially outwardly from the elongate tubular body.

Another example is a medical stent including an elongate tubular body having an intermediate body region extending between a proximal end region and a distal end region. The proximal end region defines a radially extending flange. The distal end region defines two or more anchors movable from a delivery configuration to a deployment configuration in which the two or more elongate anchors extend radially outwardly from the elongate tubular body. The medical stent also includes a polymeric covering extending over the intermediate body region.

Alternatively or additionally to any of the examples above, in another example, the polymeric covering extends over the proximal end region.

Alternatively or additionally to any of the examples above, in another example, the polymeric covering extends over at least part of the distal end region.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an example medical stent;

FIG. 2 is a schematic view of an example medical stent;

FIG. 3 is a schematic view of an example medical stent;

FIG. 4 is a schematic view of an example medical stent;

FIG. 5 is a schematic view of an example medical stent;

FIG. 6 is a schematic view of an example medical stent;

FIG. 7 is a schematic view of an example medical stent;

FIGS. 8A, 8B, 8C and 8d are schematic end views of example medical stents;

FIGS. 9A, 9B, 9C and 9d are schematic end views of example medical stents;

FIG. 10 demonstrates a possible use for an example medical stent;

FIG. 11 demonstrates a possible use for an example medical stent;

FIG. 12 demonstrates a possible use for an example medical stent;

FIG. 13 demonstrates a possible use for an example medical stent;

FIG. 14 demonstrates a possible use for an example medical stent; and

FIG. 15 is a schematic view of an example medical stent prepared for deployment.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

Stents are utilized in a variety of different body lumens, including the vasculature and various parts of the gastrointestinal system, for example. Stents may also be used in connecting one body lumen to another body lumen, such as for draining a first body lumen into a second body lumen. In some instances, one or both of the body lumens may move small distances relative to the other body lumen, meaning that a stent used to connect one body lumen to another body lumen has to be able to resist relative movement between the first body lumen and the second body lumen. A stent may be used to drain liquids from a patient's gall bladder into another part of their digestive system such as their duodenum. A stent may be used in an ERCP (endoscopic retrograde cholangiopancreatography) process. A stent may be used for hepatic duct drainage by providing a connection between the hepatic duct and a patient's stomach. A stent may be used for providing a connection between a patient's stomach and their duodenum, for example.

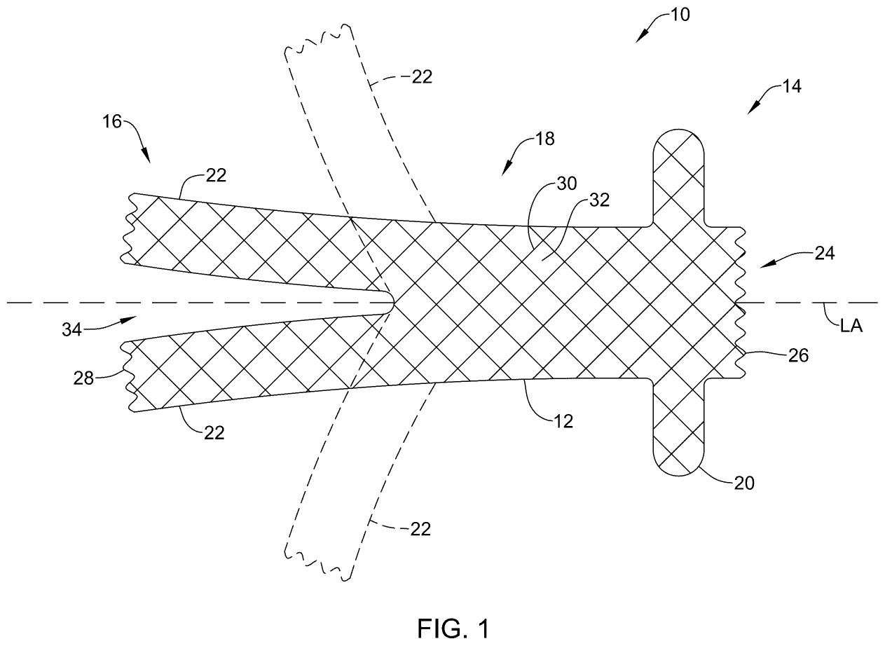

In some instances, providing artificial lumens that enable drainage of one body lumen into another body lumen may include placing and securing part of the stent to a body lumen such as an organ that is fragile as a result of a diseased state of the organ. This can mean that traditional anchor structures such as one or more radially extending flanges may cause difficulty in deploying the stent without causing further damage to the diseased organ. In some instances, subsequent removal of a stent using one or more radially extending flanges to anchor and secure the stent to the diseased organ can cause additional damage when the stent is removed. FIG. 1 is a schematic view of an example medical device, illustrated as a medical stent 10, that is adapted for use in fluidly coupling two different body lumens that may be fragile and thus less amenable to using standard mechanisms and procedures for securing the stent 10 to one or both of the body lumens, particularly when one of the body lumens may be weakened from being in a disease state.

In FIG. 1, the example medical stent 10 includes an elongate tubular body 12. While the medical stent 10 is described as generally tubular, it is contemplated that the medical stent 10 may take any cross-sectional shape desired. The elongate tubular body 12 may be considered as including a proximal end region 14, a distal end region 16, and an intervening intermediate region 18, recognizing that these designations are arbitrary, and depend on the orientation in which the medical stent 10 will ultimately be implanted. In some instances, the medical stent 10 may be a self-expanding stent (SES), configured to automatically radially expand when unconstrained, although this is not required.

In some instances, the elongate tubular body 12 may be considered as having a constant diameter throughout, including through the proximal end region 14, the distal end region 16 and the intervening intermediate region 18. In some instances, the elongate tubular body 12 may be considered as having a constant diameter when in a relaxed state. In some instances, the proximal end region 14 may be adapted to secure the elongate tubular body 12 relative to a first bodily structure such as a body lumen or even an organ. In some instances, the distal end region 16 may be adapted to secure the elongate tubular body 12 relative to a second bodily structure such as a body lumen or even an organ. In some instances, the distal end region 16 may be adapted to secure the elongate tubular body 12 relative to a second bodily structure that is damaged and/or fragile. In some instances, the distal end region 16 may be adapted to allow subsequent removal of the medical stent 10 while minimizing possible damage to the second bodily structure.

In some instances, as shown, the proximal end region 14 may include a radially extending flange 20 that may be adapted to secure the elongate tubular body 12 to a first bodily structure. Alternatively, the proximal end region 14 may instead include a flared region in which the diameter monotonically increases in the proximal direction within at least part of the proximal end region 14. In some instances, as shown, the distal end region 16 may include two or more elongate anchors 22 that are movable between a delivery configuration (shown in solid line) and a deployment configuration (shown in dashed line). In some instances, the two or more elongate anchors 22 may be constrained within the delivery configuration via a sheath or other tubular structure (not shown) that is positioned over the two or more elongate anchors 22 during delivery, and may revert to the deployment configuration in which the two or more elongate anchors 22 extend radially outwardly upon removal of the constraint. Once deployed and any constraints are removed, the two or more elongate anchors 22 may move from the delivery configuration into the deployment configuration in which each of the two or more elongate anchors 22 extend radially outwardly from the elongate tubular body 12 in order to secure the distal end region 16 in position. In some instances, the two or more elongate anchors 22 may be adapted to allow the medical stent 10 to be withdrawn proximally by pulling proximally on the medical stent 10. This may cause the two or more elongate anchors 22 to at least partially straighten, allowing the medical stent 10 to be withdrawn proximally.

In some instances, the deployment configuration of the two or more elongate anchors 22 may represent a configuration that the two or more elongate anchors 22 are biased into, and such is a configuration that the two or more elongate anchors 22 will regain when not prevented from doing so. In some instances, the deployment configuration may represent a remembered configuration. In some instances, the deployment configuration may be imparted to the two or more elongate anchors 22 via an annealing process subsequent to formation of the medical stent 10.

The medical stent 10 may include a lumen 24 that extends from a proximal end 26 of the elongate tubular body 12 to a distal end 28 of the elongate tubular body. The lumen 24 may allow for fluids and other materials to pass through the lumen 24, particularly when the medical stent 10 is being used to drain fluids and other materials from one body lumen into another body lumen. The medical stent 10 may be considered as having a longitudinal axis LA extending axially through the lumen 20. The medical stent 10 may be expandable from a first radially collapsed configuration (not shown) to a second radially expanded configuration, which may correspond to its relaxed or equilibrium state.

In some instances, the medical stent 10 may have a woven structure, fabricated from a number of filaments or struts 30 that together form the elongate tubular body 12. In some instances, the medical stent 10 may be knitted or braided with a single filament or strut interwoven with itself and defining open cells 32 extending through a wall forming the elongate tubular body 12. In some instances, the medical stent 10 may be braided with several filaments or struts interwoven together and defining open cells 32 extending along a length and around the circumference of the tubular wall of the medical stent 10. The open cells 32 may each define an opening from an outer surface of the tubular wall to an inner surface of the tubular wall (e.g., through a thickness thereof) that is free from the filaments or struts 30. Some exemplary stents including braided filaments include the WallFlex®, WALLSTENT®, and Polyflex® stents, made and distributed by Boston Scientific, Corporation. In another embodiment, the medical stent 10 may be knitted, such as the Ultraflex™ stents made by Boston Scientific, Corporation. In yet another embodiment, the medical stent 10 may be of a knotted type, such the Precision Colonic™ stents made by Boston Scientific, Corporation. In still another embodiment, the medical stent 10 may be a monolithic tubular member, for example a laser cut tubular member, such as the EPIC™ stents made by Boston Scientific, Corporation. A laser cut tubular member may have an open and/or closed cell geometry including one or more interconnected monolithic filaments or struts defining open cells 32 therebetween, with the open cells 32 extending along a length and around the circumference of the tubular wall. The open cells 32 may each define an opening from an outer surface of the tubular wall to an inner surface of the tubular wall (e.g., through a thickness thereof) that is free from the interconnected monolithic filaments or struts.

In some instances, the elongate tubular body 12 of the medical stent 10 may be made from a number of different materials such as, but not limited to, metals, metal alloys, shape memory alloys and/or polymers, as desired, enabling the medical stent 10 to be expanded into shape when accurately positioned within the body. In some instances, the material may be selected to enable the medical stent 10 to be removed with relative ease as well. For example, the elongate tubular body 12 of the medical stent 10 can be formed from alloys such as, but not limited to, nitinol and Elgiloy®. Depending on the material selected for construction, the medical stent 10 may be self-expanding or require an external force to expand the medical stent 10. In some embodiments, composite filaments may be used to make the medical stent 10, which may include, for example, an outer shell or cladding made of nitinol and a core formed of platinum or other radiopaque material. It is further contemplated the elongate tubular body 12 of the medical stent 10 may be formed from polymers including, but not limited to, polyethylene terephthalate (PET). In some instances, the filaments of the medical stent 10, or portions thereof, may be bioabsorbable or biodegradable, while in other instances the filaments of the medical stent 10, or portions thereof, may be biostable.

In some instances, the two or more elongate anchors 22 may be at least partially defined by virtue of a plurality of more elongate voids 34, such as two or more elongate voids 34, although only a single elongate void 34 is visible in this view. Each of the two or more elongate voids 34 may be considered as extending from a terminal end (e.g., the distal end 28) in a direction parallel to, or at least substantially parallel to, the longitudinal axis LA. Substantially parallel may be defined as within ten percent of parallel, for example. In some instances, while not shown, one or more of the elongate voids 34 may not be parallel or substantially parallel to the longitudinal axis LA, but may instead be disposed at an acute angle with respect to the longitudinal axis LA. In some instances, the distal end region 16 may include a second elongate void 32 that is circumferentially spaced about 180 degrees about the elongate tubular body 12. The distal end region 16 may include two, three, four or more elongate voids 34, for example. Each of the elongate voids 34 may have the same length. In some instances, one or more of the elongate voids 34 may have a different length. As a result, in some instances, one or more of the two or more elongate anchors 22 may have a different length than others of the two or more elongate anchors 22.

The elongate voids 34 may be formed in any suitable manner. In some instances, the elongate tubular body 12 may be formed, and the elongate voids 34 may be subsequently cut into the elongate tubular body 12, removing portions of the elongate tubular body 12. As an example, the elongate voids 34 may be laser cut into the elongate tubular body 12 subsequent to forming the tubular body 12 (e.g., subsequent to braiding/weaving the filaments 26 of the tubular body 12). The elongate voids 34 may be mechanically cut into the elongate tubular body 12. In some instances, the elongate tubular body 12 may be formed to include the elongate voids 34 as the tubular body 12 is formed. In some instances, one or more of the elongate anchors 22 may have a braid pattern that is different from that of the intermediate body portion 18. As an example, one or more filaments that would otherwise cross where one or more of the elongate voids 34 are may be braided or otherwise formed into part of one of the elongate anchors 22. In some instances, each of the two or more elongate anchors 22 are braided independently from braiding the rest of the elongate tubular body 12.

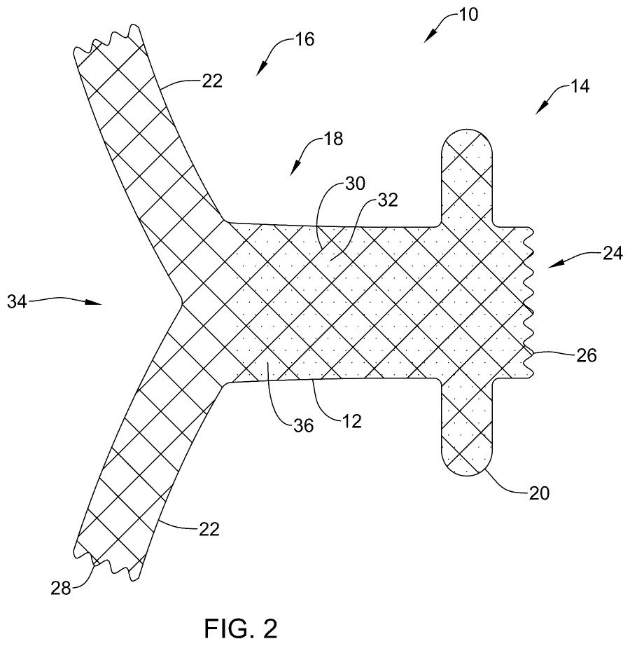

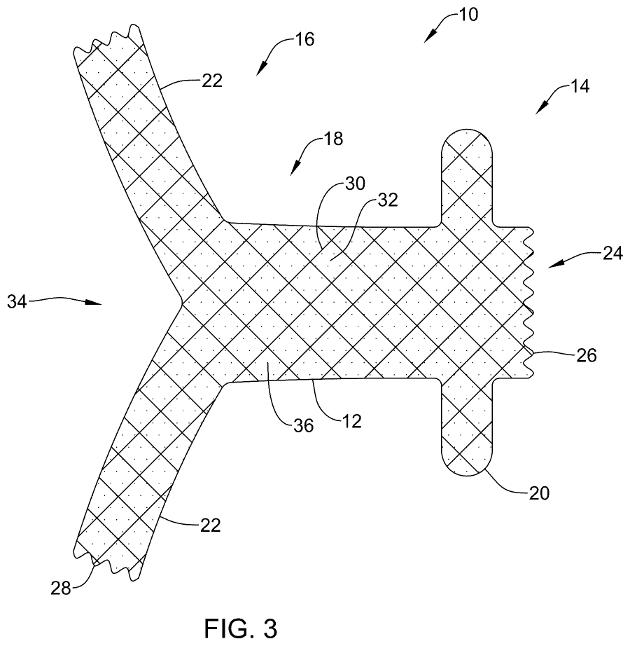

In some instances, an inner and/or outer surface of the elongate tubular body 12 may be entirely, substantially, or partially, covered with a polymeric coating that facilitates use of the medical stent 10 as a drain from draining fluid and other materials from one body lumen to another body lumen. FIG. 2 is a schematic view of the example medical stent 10, showing a polymeric covering 36 that is illustrated as a dotted pattern over the elongate tubular body 12. The polymeric covering 36 may extend across and/or occlude one or more, or a plurality of the open cells 32 defined by the struts or filaments 30. As shown in FIG. 2, the polymeric covering 36 extends over the proximal end region 14 as well as the intermediate body region 18, but does not extend over the distal end region 16 and does not extend over the elongate anchors 22. As shown in FIG. 3, the polymeric covering 36 extends over the proximal end region 14, the intermediate body region 18 as well as extending over the distal end region 16. As a result, the polymeric covering 36 extends over the elongate anchors 22, but does not extend over the elongate voids 34.

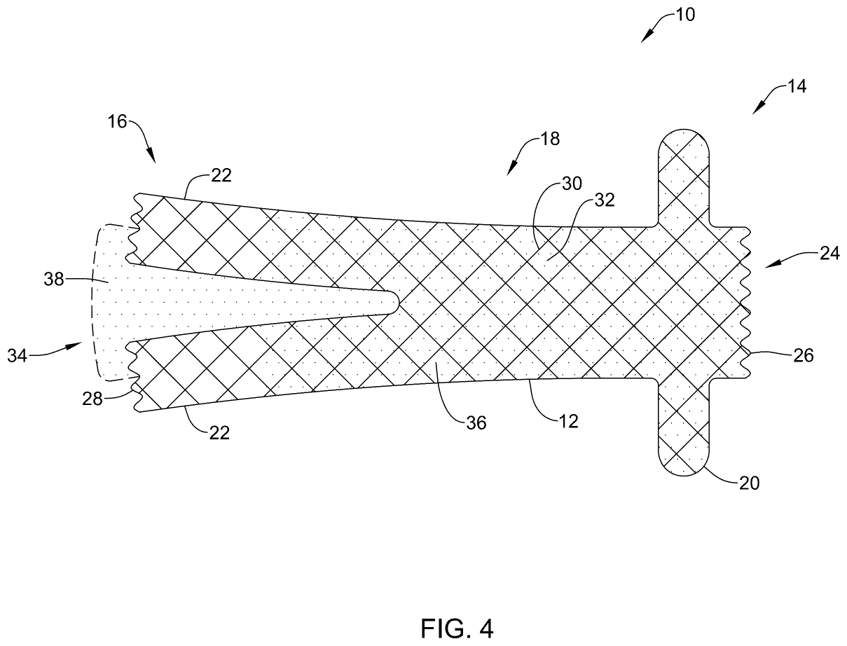

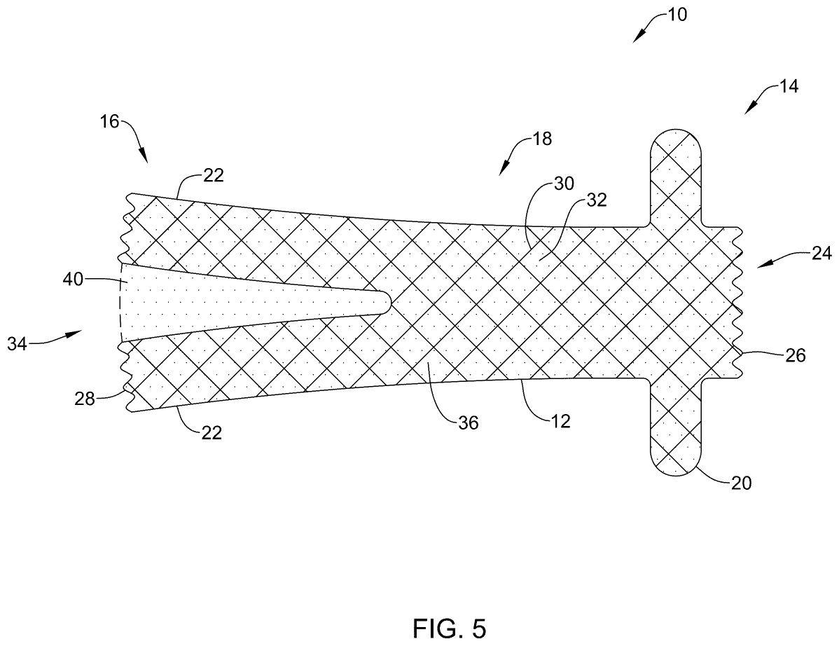

FIG. 4 is a schematic view of the example medical stent 10, showing that the polymeric covering 36 includes an extension 38 of the polymeric covering 36 over the intermediate body region 18 that extends beyond the distal end 28 of the elongate tubular body 12, thereby extending a channel formed by the lumen 24. FIG. 5 is a schematic view of the example medical stent 10, showing that the polymeric covering 36 includes a loose skirt 40 that allows the elongate anchors 22 to move while still extending a channel formed by the lumen 24 in order to allow for unimpeded drainage through the lumen 24.

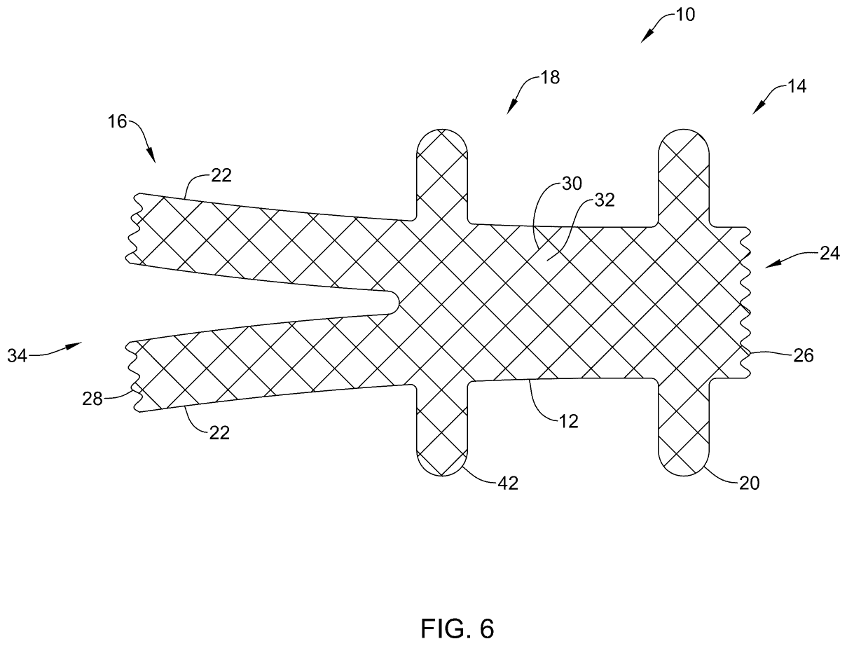

In some instances, as shown in FIG. 6, the example medical stent 10 may include not only the radially extending flange 20 within the proximal end region 14, but may also include a second radially extending flange 42 that is disposed distal of the radially extending flange 20. In some instances, the second radially extending flange 42 may be considered as extending within the intermediate body region 18. Including the second radially extending flange 42 may further facilitate antimigration of the example medical stent 10. While no polymeric covering 36 is shown in FIG. 6, it will be appreciated that the example medical stent 10 may include a polymeric covering 36 as shown in any of FIGS. 2 through 5.

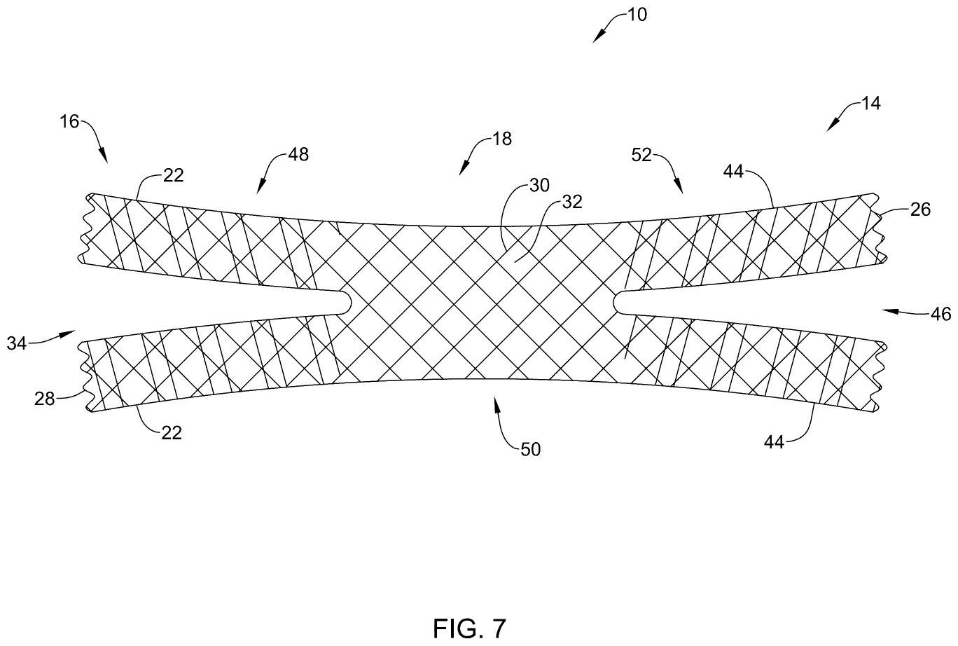

FIG. 7 is a schematic view of the example medical stent 10 in which the proximal end region 14 does not include the flange 22, but instead includes a second two or more elongate anchors 44 that are at least partially defined by a two or more elongate voids 46 (only one of the elongate voids 46 is visible in this view). In some instances, the second two or more elongate anchors 44 are adapted to move between a delivery configuration (as shown) and a deployment configuration in which the two or more elongate anchors 44 extend radially outwardly upon removal of the constraint. Once deployed and any constraints are removed, the two or more elongate anchors 44 may move from the delivery configuration into the deployment configuration in which each of the two or more elongate anchors 44 extend radially outwardly from the elongate tubular body 12 in order to secure the proximal end region 14 in position.

In some instances, the deployment configuration of the two or more elongate anchors 44 may represent a configuration that the two or more elongate anchors 44 are biased into, and such is a configuration that the two or more elongate anchors 44 will regain when not prevented from doing so. In some instances, the deployment configuration may represent a remembered configuration. In some instances, the deployment configuration may be imparted to the two or more elongate anchors 44 via an annealing process subsequent to formation of the medical stent 10.

In some instances, as shown, the two or more elongate anchors 22 may have a braiding pattern 48 that is different from a braiding pattern 50 within the intermediate body portion 18. In some instances, as shown, the two or more elongate anchors 44 may have a braiding pattern 52 that is different from the braiding pattern 48 used for the two or more elongate anchors 22 as well as being different from the braiding pattern 50 used for the intermediate body portion 18. In some instances, the braiding pattern 48 and the braiding pattern 52 may be the same, although different from the braiding pattern 50. In some instances, one or more of the two or more elongate anchors 22 may have a different braiding pattern compared to others of the two or more elongate anchors 22. In some instances, one or more of the two or more elongate anchors 44 may have a different braiding pattern compared to others of the two or more elongate anchors 44.

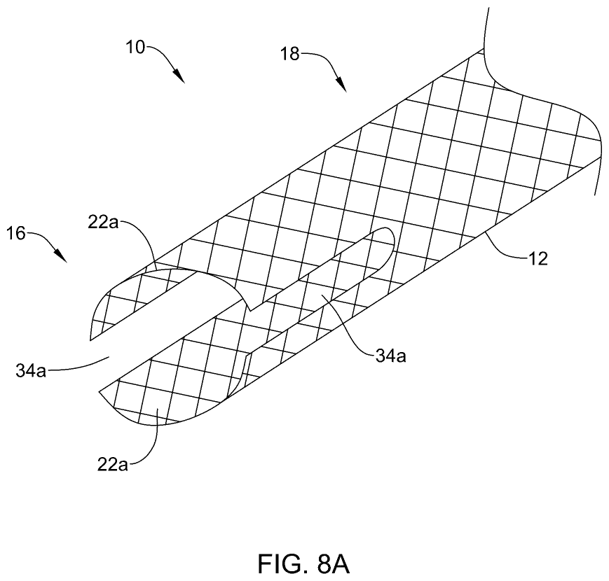

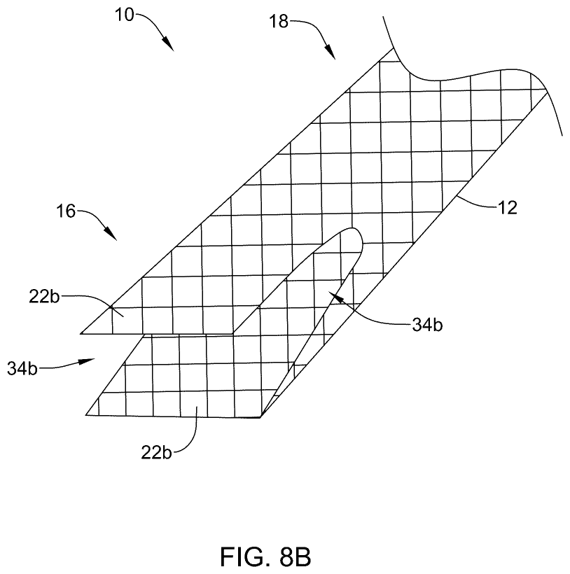

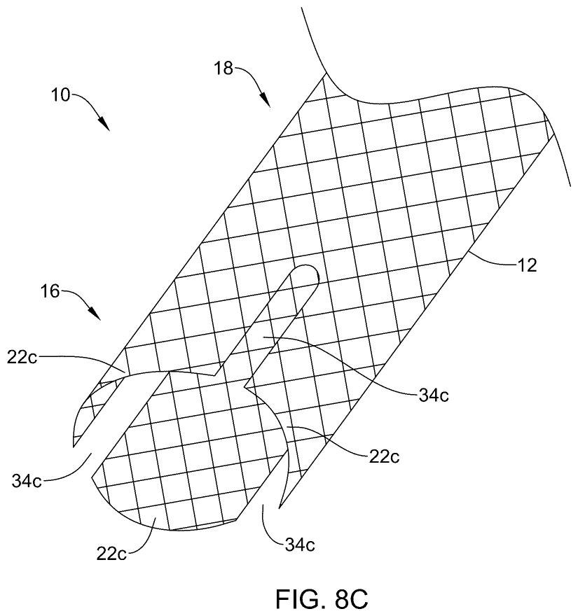

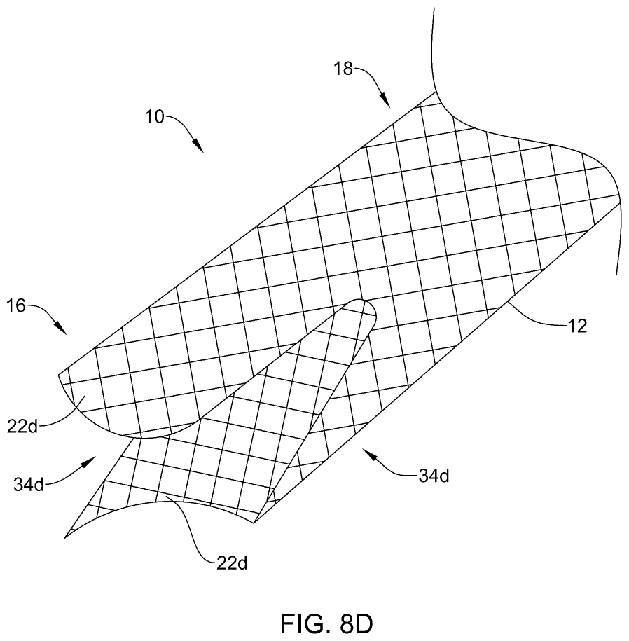

The distal end region 16 of the example medical stent 10 may take a variety of forms. In some instances, modifying the form of the distal end region 16 may facilitate deployment of the distal end region within various anatomies. As shown in FIG. 8A, the distal end region 16 may include a total of two elongate anchors 22a, separated from each other by two elongate voids 34a. While two elongate anchors 22a and two elongate voids 34a are shown, it will be appreciated that the distal end region 16 may include any number of elongate anchors 22a and elongate voids 34a. The elongate anchors 22a may be seen as having a curved profile that matches a curved profile of the intermediate body region 18. When viewed from a position exterior to the example medical stent 10, the elongate anchors 22a may be considered as having a convex profile that matches a convex profile of the intermediate body region 18. In FIG. 8B, the distal end region 16 includes a pair of two elongate anchors 22b, separated from each other by two elongate voids 34b. While two elongate anchors 22b and two elongate voids 34b are shown, it will be appreciated that the distal end region 16 may include any number of elongate anchors 22b and elongate voids 34b. The elongate anchors 22b can be seen as having a planar or flat profile that is different from the curved profile of the intermediate body region 18.

As shown in FIG. 8C, the distal end region 16 may include a total of two elongate anchors 22c, separated from each other by two elongate voids 34c. While two elongate anchors 22c and two elongate voids 34c are shown, it will be appreciated that the distal end region 16 may include any number of elongate anchors 22c and elongate voids 34c. The elongate anchors 22c may be seen as having a curved profile that matches a curved profile of the intermediate body region 18. When viewed from a position exterior to the example medical stent 10, the elongate anchors 22c may be considered as having a convex profile that matches a convex profile of the intermediate body region 18.

As shown in FIG. 8D, the distal end region 16 may include two elongate anchors 22d, separated from each other by two elongate voids 34d. While two elongate anchors 22d and two elongate voids 34d are shown, it will be appreciated that the distal end region 16 may include any number of elongate anchors 22d and elongate voids 34d. The elongate anchors 22d may be seen as having a curved profile that is opposite a curved profile of the intermediate body region 18. When viewed from a position exterior to the example medical stent 10, the elongate anchors 22d may be considered as having a concave profile that is opposite a convex profile of the intermediate body region 18.

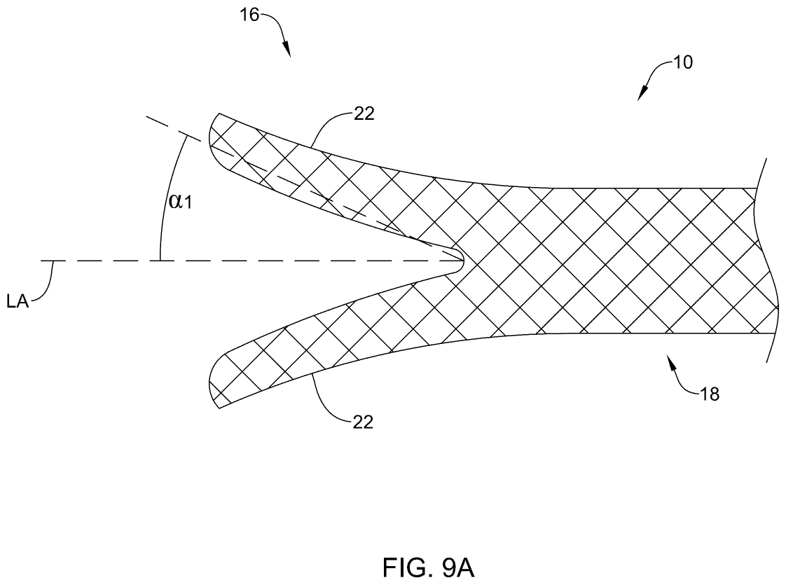

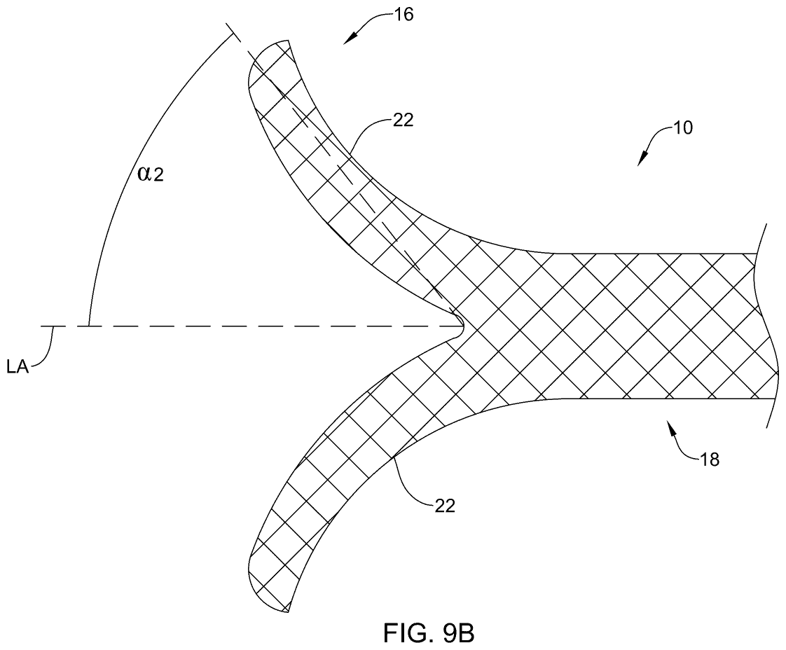

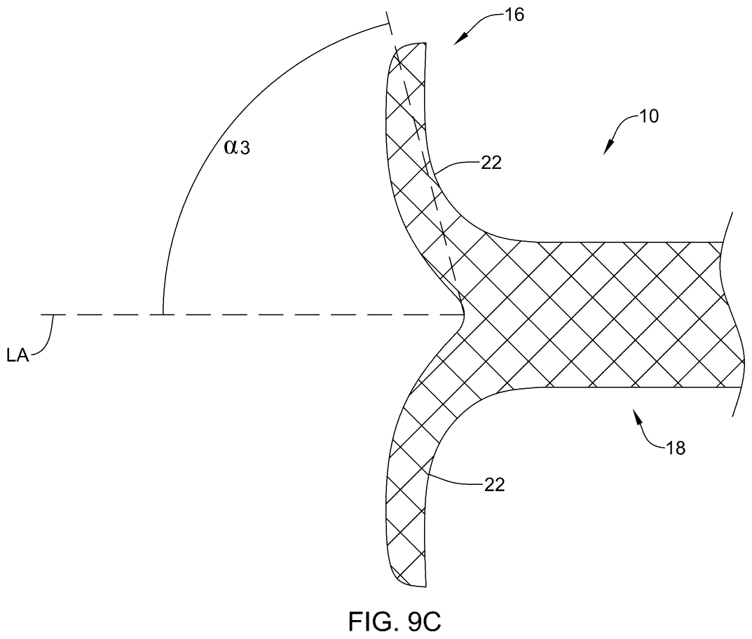

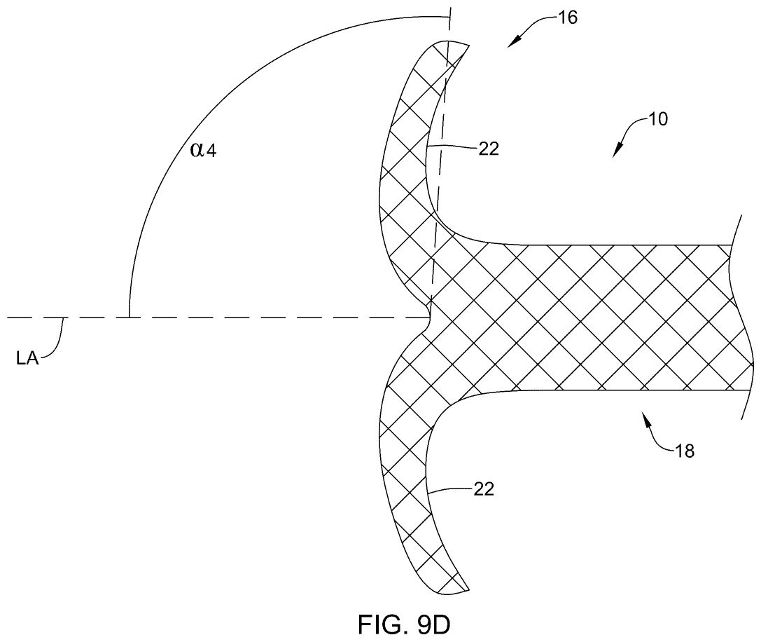

FIGS. 9A through 9D are schematic views of the example medical stent 10, illustrating how the elongate anchors 22 may be provided with a variety of different deployment configurations. In FIG. 9A, the elongate anchors 22 curve radially outwardly a relative short distance, with each of the elongate anchors 22 forming an angle α1 with the longitudinal axis LA that is an acute angle, and is about 30 degrees In FIG. 9B, the elongate anchors 22 curve radially outwardly a distance greater than that shown in FIG. 9a, with each of the elongate anchors 22 forming an angle α2 with the longitudinal axis LA that is an acute angle, and is about 45 degrees. In FIG. 9C, the elongate anchors curve radially outwardly a greater distance, with each of the elongate anchors 22 forming an angle α3 with the longitudinal axis LA that is approaching a right angle. In FIG. 9D, the elongate anchors 22 curve radially outwardly an even greater distance, with each of the elongate anchors 22 forming an angle α4 that is an obtuse angle, i.e., greater than 90 degrees. In identifying these angles, it will be appreciated that these angles are merely approximations, given the curvature of the elongate anchors 22.

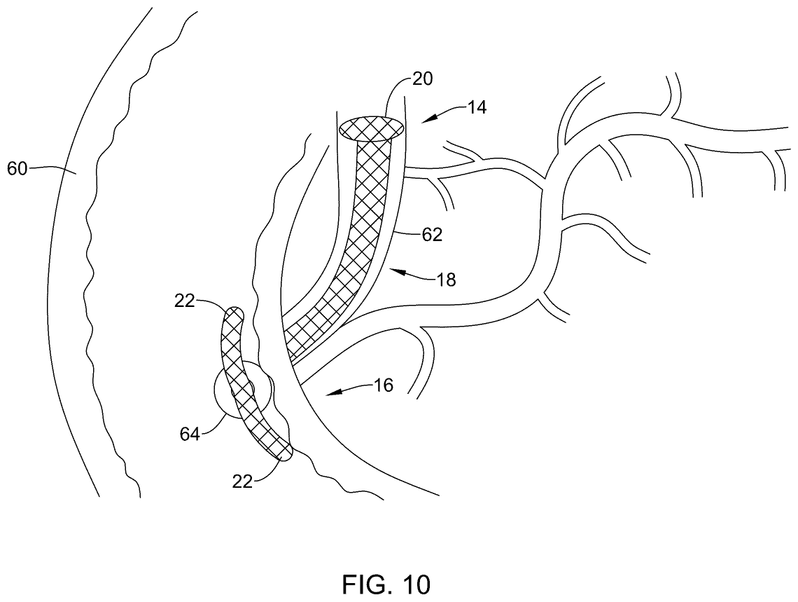

FIG. 10 is a schematic view showing the example medical stent 10 deployed within a patient's anatomy for an ERCP (endoscopic retrograde cholangiopancreatography) process. The anatomy includes a duodenum 60, a bile duct 62 and the Papilla of Vater 64 where the bile duct 62 (and the pancreatic duct, not shown) joins the duodenum 60. The distal end region 16 of the medical stent 10 is deployed within the duodenum 60 proximate the Papilla of Vater 64. The proximal end region 14, including the radially extending flange 20, is disposed within the bile duct 62. The radially extending flange 20 helps to secure the medical stent 10 within the bile duct 62. It will be appreciated that this location is reached from within the duodenum 60, meaning that the medical stent 10 was delivered and implanted in a backwards orientation, where the proximal end region 14 was actually distal-most, and the distal end region 16 was actually proximal-most. The medical stent 10 is held in place, and allows drainage through the bile duct 62 (from the gall bladder, not identified) and into the duodenum 60.

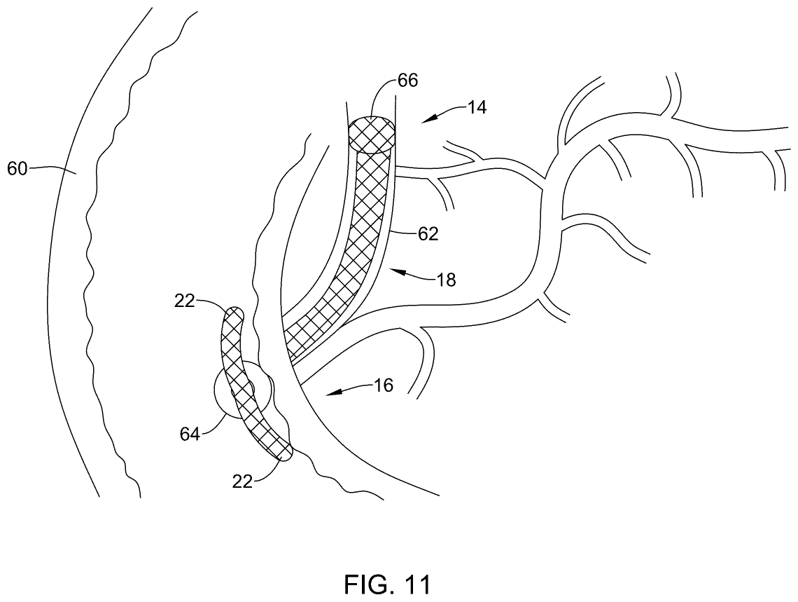

FIG. 11 also demonstrates an ERCP (endoscopic retrograde cholangiopancreatography) process, but in this case the proximal end region 14 does not include the radially extending flange 20, but instead includes a flared region 66 in which the outer diameter of a portion of the proximal end region 14 is monotonically increasing. The distal end region 16 of the medical stent 10 is deployed within the duodenum 60 proximate the Papilla of Vater 64. The proximal end region 14, including the radially extending flared region 66, is disposed within the bile duct 62. The flared region 66 helps to secure the medical stent 10 within the bile duct 62. It will be appreciated that this location is reached from within the duodenum 60, meaning that the medical stent 10 was delivered and implanted in a backwards orientation, where the proximal end region 14 was actually distal-most, and the distal end region 16 was actually proximal-most. The medical stent 10 is held in place, and allows drainage through the bile duct 62 (from the gall bladder, not identified) and into the duodenum 60.

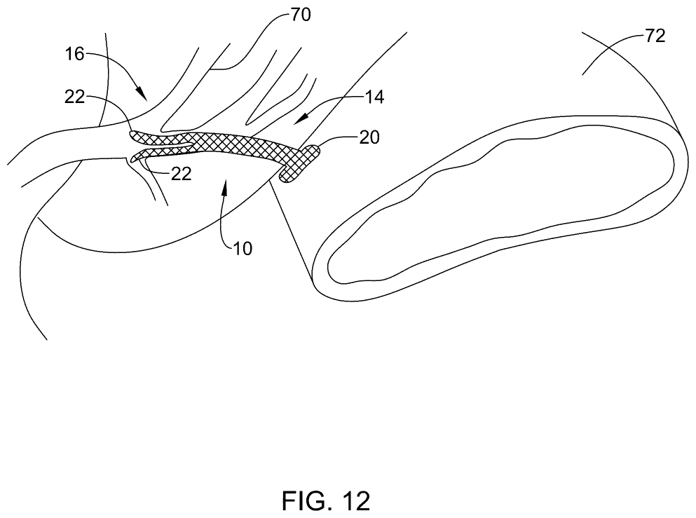

FIG. 12 demonstrates use of the medical stent 10 in a Hepaticogastrostomy (HGS) procedure, which is an effective biliary drainage procedure in adult cases with difficult biliary access, which may be performed under endoscopic guidance (EUS-HGS). The HGS procedure involves draining a hepatic duct 70 into the stomach 72. The distal end region 16, including the elongate anchors 22, are disposed within the hepatic duct 70. The proximal end region 14, including the radially extending flange 20, is disposed within the stomach 72. The elongate anchors 22 help keep the medical stent 10 from migrating proximally into the stomach 72. The radially extending flange 20 helps keep the medical stent from migrating distally out of the stomach 72. In some instances, the elongate anchors 22 may be adapted to have a remembered configuration in which they extend radially outwardly to an angle of less than 90 degrees, which may provide a better fit within the hepatic duct 70.

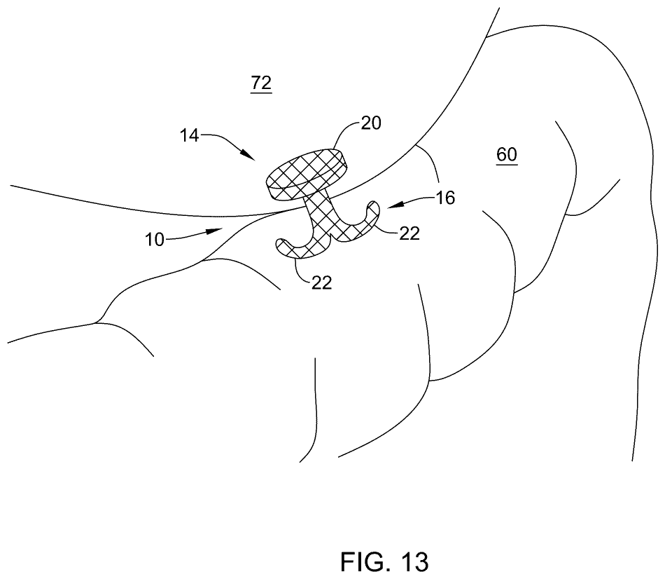

FIG. 13 demonstrates use of the medical stent 10 in a procedure in which a fluid pathway is formed between the stomach 72 and the duodenum 60. In some instances, this may represent use of the medical stent 10 in a gastric bypass procedure in which the medical stent 10 serves as an anastomosis between the stomach 72 and the duodenum 60, thereby causing stomach contents including food to bypass an upper portion of the duodenum 60. As shown, the proximal end region 14 of the medical stent 10 is disposed within the stomach 72 and the distal end region 16 of the medical stent 10 is disposed within the duodenum 60. The radially extending flange 20 helps to keep the medical stent 10 from migrating into the duodenum 60 while the elongate anchors 22 help to keep the medical stent 10 from migrating into the stomach 72.

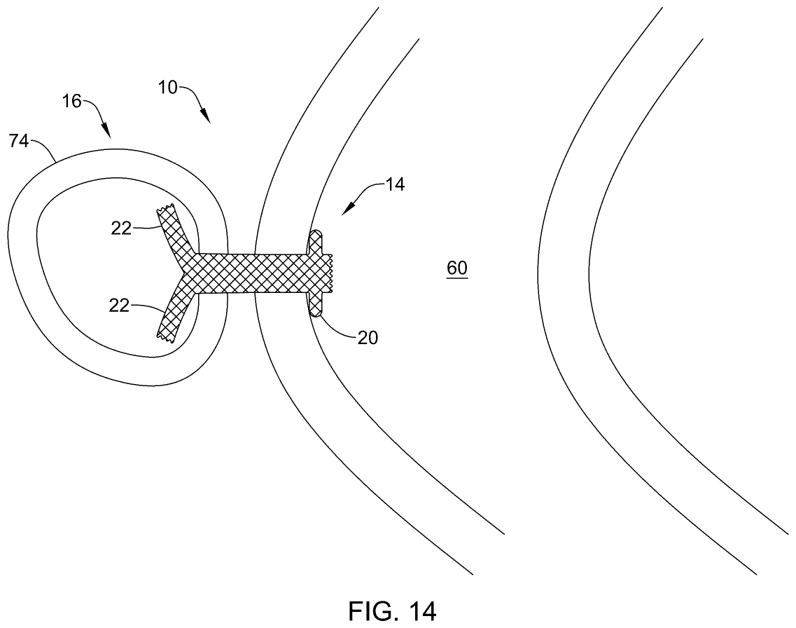

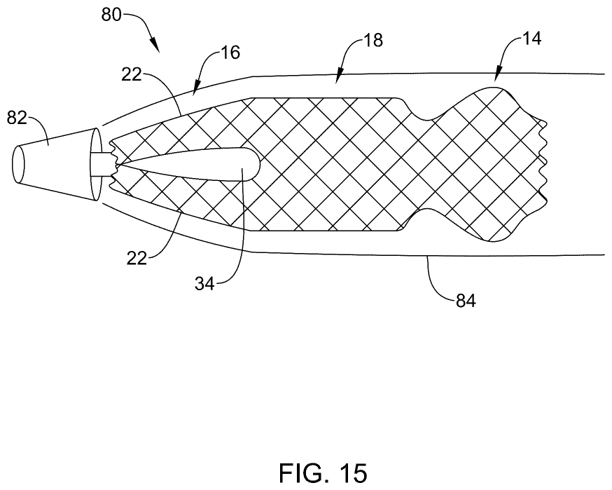

FIG. 14 demonstrates use of the medical stent 10 in a procedure whereby a gall bladder 74 is fluidly coupled with the duodenum 60 in order to drain fluids and other materials from the gall bladder 74. The medical stent 10 may be advanced through the duodenum 60, and through a first aperture formed in the wall of the duodenum 60 and through a second aperture formed in the wall of the gall bladder 74. The radially extending flange 20 helps to secure the medical stent 10 relative to the duodenum and the elongate anchors 22 help to secure the medical stent 10 relative to the gall bladder 74. The medical stent 10 may be advanced through the duodenum 60 and into the gall bladder 74 using a delivery device shown in FIG. 15.

FIG. 15 is a schematic view of the example medical stent 10 disposed within a delivery device 80. The delivery device 80 includes a distal tip 82 and a sheath 84 extending proximally from the distal tip 82. The sheath 84 keeps the medical stent 10 in a collapsed configuration for delivery. By virtue of the elongate voids 34 that separate the elongate anchors 22, it will be appreciated that the distal end region 16 of the medical stent 10 may be collapsed down to a smaller diameter than would otherwise be possible without the elongate voids 34. Making the distal tip 82 smaller means that the apertures that need to be formed within the wall of the duodenum 60 and the gall bladder 74, for example, can be smaller as well. This means less possible damage to the duodenum 60 and especially to the gall bladder 74, as the gall bladder 74 may be in a weakened or fragile state due to whatever disease is necessitating draining the gall bladder 74.

The materials that can be used for the various components of the medical stent(s), the mandrel, and the various elements thereof disclosed herein may include those commonly associated with medical devices and mandrels. For simplicity purposes, the following discussion refers to the apparatus. However, this is not intended to limit the devices and methods described herein, as the discussion may be applied to other elements, members, components, or devices disclosed herein, such as, but not limited to, the medical stent, the mandrel, the filaments, the anti-migration loops, the covering, and/or elements or components thereof.

In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical stent, comprising:
an elongate tubular body having an intermediate body region extending between a proximal end region and a distal end region;
the proximal end region defining a radially extending flange that is adapted to secure the elongate tubular body relative to a first bodily structure;
the distal end region defining two or more elongate anchors movable from a delivery configuration to a deployment configuration in which the two or more elongate anchors extend radially outwardly from the elongate tubular body, the two or more elongate anchors adapted to secure the elongate tubular body relative to a second bodily structure when in the deployment configuration.

2. The medical stent of claim 1, wherein the distal end region comprises two or more elongate voids disposed between the two or more elongate anchors.

3. The medical stent of claim 1, wherein the deployment configuration represents a remembered configuration for the two or more elongate anchors.

4. The medical stent of claim 1, wherein each of the two or more elongate anchors are constrained during delivery to maintain the two or more elongate anchors in the delivery configuration, and each of the two or more elongate anchors are adapted to move into the deployment configuration upon removal of the constraint.

5. The medical stent of claim 1, wherein the two or more elongate anchors have a curved profile matching a curved profile of the intermediate body region of the elongate tubular body when in the delivery configuration.

6. The medical stent of claim 1, wherein the two or more elongate anchors have a curved profile curving in an opposite direction from a curved profile of the intermediate body region of the elongate tubular body when in the delivery configuration.

7. The medical stent of claim 1, wherein the two or more elongate anchors have a planar profile different from a curved profile of the intermediate body region of the elongate tubular body when in the delivery configuration.

8. The medical stent of claim 1, wherein at least one of the two or more elongate anchors have a braid pattern different from that of the intermediate body region of the elongate tubular body.

9. The medical stent of claim 1, further comprising a polymeric covering extending over the intermediate body region of the elongate tubular body.

10. The medical stent of claim 9, wherein the polymeric covering extends over the at least two elongate anchors.

11. The medical stent of claim 9, wherein the polymeric covering extends between the at least two elongate anchors.

12. The medical stent of claim 1, further comprising a second radially extending flange distal of the radially extending flange within the proximal end region.

13. The medical stent of claim 1, wherein the elongate tubular body comprises a braided stent body.

14. A medical stent, comprising:
an elongate tubular body having an intermediate body region extending between a proximal end region and a distal end region;
the proximal end region defining a proximal end anchor structure;
the distal end region defining two or more anchors movable from a delivery configuration to a deployment configuration in which the two or more elongate anchors extend radially outwardly from the elongate tubular body.

15. The medical stent of claim 14, wherein the proximal end anchor structure comprises a radially extending flange.

16. The medical stent of claim 14, wherein the proximal end anchor structure comprises a radially extending flange.

17. The medical stent of claim 14, wherein the proximal end anchor structure comprises an additional two or more elongate anchors movable from a delivery configuration to a deployment configuration in which the additional two or more elongate anchors extend radially outwardly from the elongate tubular body.

18. A medical stent, comprising:
an elongate tubular body having an intermediate body region extending between a proximal end region and a distal end region;
the proximal end region defining a radially extending flange;
the distal end region defining two or more anchors movable from a delivery configuration to a deployment configuration in which the two or more elongate anchors extend radially outwardly from the elongate tubular body; and
a polymeric covering extending over the intermediate body region.

19. The medical stent of claim 18, wherein the polymeric covering extends over the proximal end region.

20. The medical stent of claim 18, wherein the polymeric covering extends over at least part of the distal end region.
